(19) United States
(12) Patent Application Publication
(10) Pub. No.: US 2007/0162034 A1
(43) Pub. Date: Jul. 12, 2007

(54) Annular repair method and device

(76) Inventors: John Kostuik, Baltimore, MD (US); Patrick Connolly, Worchester, MA (US); Kevin R. Strauss, Columbia, MD (US); Richard W. Woods, Catonsville, MD (US); Mary Patricia Hamburger, Herndon, VA (US); Andy Rock, Spring Grove, PA (US)

Correspondence Address: PERRY E. VAN OVER & ASSOCIATES, PLLC, P.O. BOX 399, FAIRFAX, VA, 22038, US

(21) Appl. No.: 11/498,036

(22) Filed: Aug. 3, 2006

Related U.S. Application Data
(60) Provisional application No. 60/705,147, filed on Aug. 4, 2005.

(51) Int. Cl. A61B 17/16

(52) U.S. Cl. 606/80

(57) ABSTRACT

A method of repairing an annulus of an intervertebral disc includes excising, with a cutting tool, a damaged area of the annulus, thereby creating a space in the annulus, the space being defined by first and second annulus ends; making a partial incision at the first annulus end generally adjacent to the excised area, creating a flap of annulus; stretching the flap across the space, and attaching the flap to the second annulus end.

This application claims the benefit of the earlier filing date of U.S. Provisional Application No. 60/705,147, filed on Aug. 4, 2005, which is incorporated by reference herein in its entirety.

### BACKGROUND

1. Technical Field

This invention relates generally to orthopedic spine surgery and in particular to devices, systems and methods for use in the repair of the annulus of the intervertebral disc.

2. Background Art

The use of discectomy procedures for the repair of herniated intervertebral discs in the human spinal column is well documented in the literature and techniques for performing these procedures using a variety of methods have been well described in the prior art. In these procedures the disc is excised, the herniation removed along with a portion of the nucleus pulposus and the patient's symptoms typically abate. The application of these procedures has become widely accepted throughout the medical community and they have proven to be highly successful clinically.

Many techniques have been described and are currently used to access and then incise disc herniations, and even more methods, and numerous devices, are available for the removal of the herniation and subsequent nuclear material from the disc space, including the use of mechanical devices such as curettes and rongeurs, ultrasonic devices, mechanical shavers and heat generating equipment. Whenever such methods and devices are used the remaining void in the annular wall remains unfilled. Few materials or devices have been developed which can be reliably utilized to close this void. Consequently, to prevent the later escape of nuclear material from the remaining hole, surgeons have taken to removing more of the disc material than is necessary so that no material remains near the opening. This appears to address the clinical need, but the effect on the residual disc is unclear and is suspected to be negative, possibly weakening the remaining structure and increasing the potential for later expulsion of the nucleus that remains.

More recently there has been considerable focus on this problem of closing the void and a number of devices have been developed to try and address this clinical need. This includes some configurations that thread into the opening or try to attach to the bone around the void, but this is difficult due to the dynamic nature of the interface, whereby the vertebrae move independently of each other making it difficult to establish a rigid bridge across the opening or void.

There is, therefore, a demonstrated need for an improved method and surgical technique for excising, then repairing these disc herniations in a simple manner that can seal the annulus and prevent the later expulsion of nucleus pulposus and subsequent recurrence of the herniation. The prior art methods and devices have not been capable of providing these features.

### SUMMARY OF THE DISCLOSURE

It is a general object of the present invention to provide a method for spine surgery to repair and seal a disc annulus.

It is a further object of the present invention to provide a method for spine surgery to repair and seal a disc annulus due to a herniation or other damage.

It is a further object of this invention to provide a minimally invasive method whereby a cannula or tool is used for performing spine surgery to repair and seal a disc annulus.

It is a further object of this invention to provide a device for performing minimally invasive spine surgery to repair and seal a disc annulus.

It is a further object of this invention to provide a method for performing spine surgery to repair and seal a disc annulus using a portion of the patient's injured disc which is still partially attached.

It is a further object of this invention to provide a method for performing spine surgery to repair and seal a disc annulus using an autograft of the patient's annulus.

In accordance with the principles of the present invention, these objectives are achieved by providing a method of repairing an annulus of an intervertebral disc. The method includes excising, with a cutting tool, a damaged area of the annulus, thereby creating a space in the annulus, the space being defined by first and second annulus ends; making a partial incision at the first annulus end generally adjacent to the excised area, creating a flap of annulus; stretching the flap across the space; and attaching the flap to the second annulus end.

In accordance with another aspect of the invention, a method of repairing an annulus of an intervertebral disc includes excising, with a cutting tool, a damaged area of the annulus, thereby creating a space in the annulus, the space being defined by first and second annulus ends; and attaching a connector between the first and second ends so that the connector spans the space.

In accordance with yet another aspect of the invention, a tool is provided for cutting and removing a portion of an annulus of an intervertebral disc. The tool includes an elongated body having a bore there-through. A cutting structure is associated with the elongated body and is constructed and arranged to cut a portion of the annulus. A structure is movable within the bore and has an end that is constructed and arranged to engage and thereby remove the portion cut by the cutting structure.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The foregoing and other features of the disclosed embodiments will become apparent to one skilled in the art, relates upon consideration of the following description, with reference to the accompanying drawings, wherein:

FIG. 1 is a transverse sectional view through a diseased vertebral disc showing a conventional herniation at the posterior edge.

FIG. 2 is a transverse sectional view through the diseased vertebral disc of FIG. 1 showing a cannula or a cutting tool of the present invention used to excise the bulge.

FIG. 3 is a transverse sectional view through the diseased vertebral disc of FIG. 2 showing a resected portion of an annular wall and a partial surgical cut.

FIG. 3A is an enlarged view of the portion encircled at A in FIG. 3 showing the herniated area and the surgical cut made adjacent to the resected annulus.

FIG. 4 is a transverse sectional view through the diseased vertebral disc of FIG. 3 showing an annular flap folded over to close a gap.

FIG. 4A is an enlarged view of the portion encircled at A in FIG. 4 showing the herniated area and the annular flap pulled across the gap and stitched.

FIG. 5 is a front view of a cutting tool provided in accordance with the principles of the present invention, shown with a collar in a first position.

FIG. 6 is a front view of a cutting tool provided in accordance with the principles of the present invention, shown with the collar in a second, blade-extending position and a barb structure for excising an annulus portion.

FIG. 7 is a front view of a cutting tool provided in accordance with another embodiment of the present invention.

FIG. 8 is a top view of the cutting tool of FIG. 7.

FIG. 9 is an enlarged view of a connector, in accordance with the principles of the invention, spanning a space defined by removal of a damaged area of an annulus.

### DETAILED DESCRIPTION OF PREFERRED EMBODIMENTS

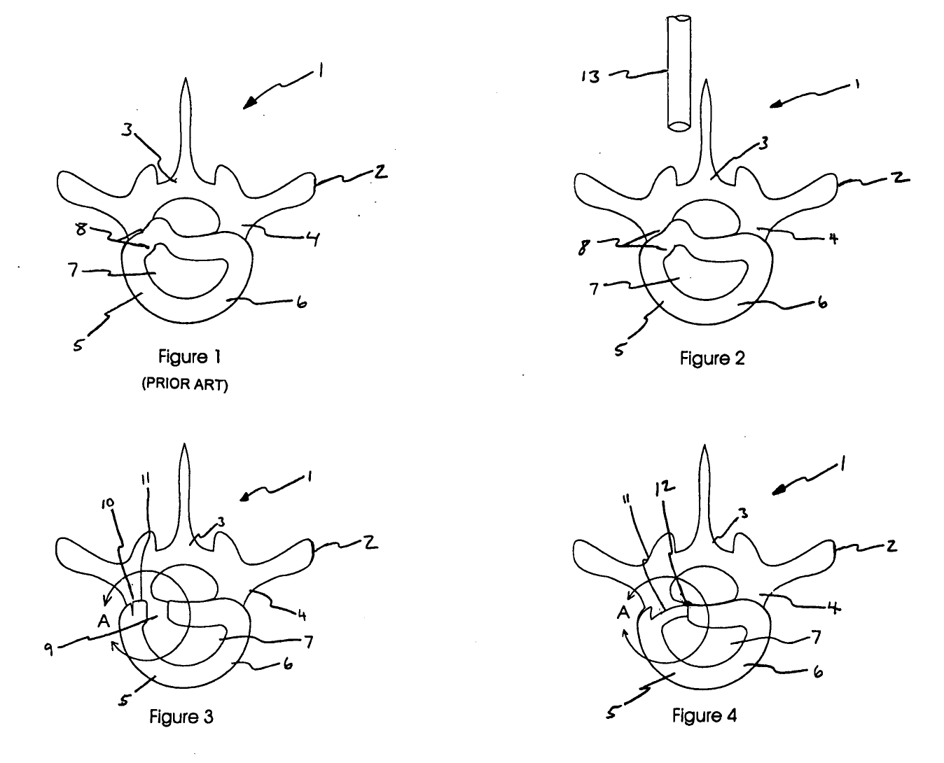

With reference to FIGS., 1-4, a method repairing the intervertebral disc annulus damaged as a result of a herniation or damage to the disc will be described in accordance with the principles of the present invention. FIGS. 1-4 illustrate a transverse sectional view of the vertebral body, generally indicated at 1, and the disc 5. Common to FIGS. 1-4 is the relevant anatomy of the transverse sectional view including the transverse process 2, lamina 3, pedicle 4, annulus fibrosus 6 of the disc, and nucleus pulposus 7 of the disc. FIG. 1 shows the vertebral body 1, whereby the disc 5 is herniated 8. FIG. 2 illustrates a minimally invasive cannula 13, positioned over the herniation 8 and passing by the lamina 3. A cutting tool (not shown in FIG. 2) can be passed through the cannula 13, to excise the disc annulus 6. Also in FIG. 2, using the minimally invasive cannula 13, subsequent instruments can be passed there-through to remove a portion of the disc nucleus 7.

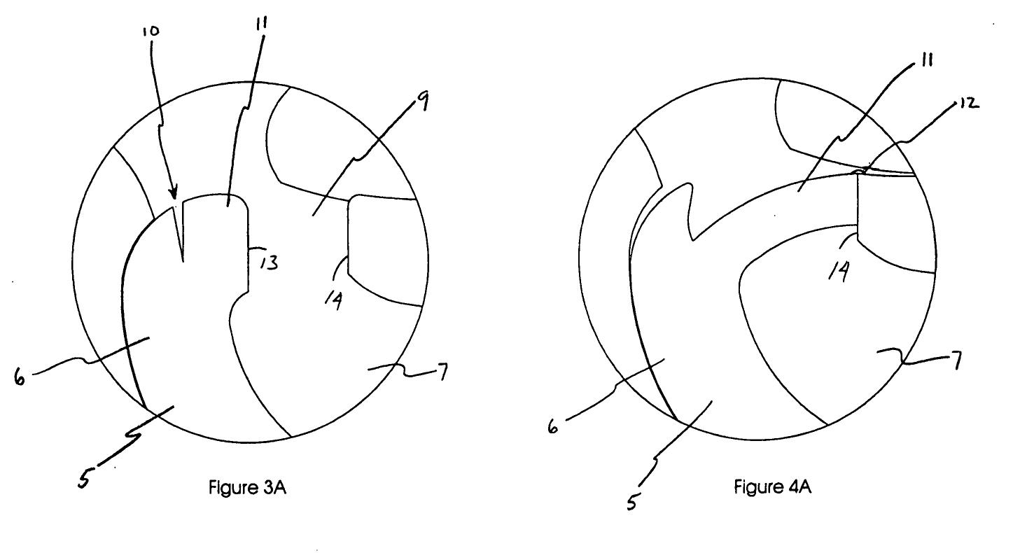

FIG. 3 illustrates a gap or space 9 in the disc annulus 6 created by the surgeon. FIG. 3 also shows a surgical incision 10 made in the disc annulus 6, adjacent to the space 9, such that the distance from the edge of the space 9 to the surgical incision 10 creates a portion of disc tissue or “flap” 11 that can be stretched across the space 9. Further, the surgical incision 10 is not cut so deep as to invade the disc nucleus 7. FIG. 4 illustrates the flap 11 of the disc annulus 6 being stretched across the space 9 and securely attached 12 to the other side of the disc annulus 6. FIG. 4 also shows where the flap 11, is attached (the attachment indicated at 12) to its adjacent annulus 6 to close the disc space 9 and prevent any further herniation of the disc nucleus 7.

FIGS. 3A and 4A show enlarged views of the portions encircled at A in FIGS. 3 and 4, respectively, for clarification. FIG. 3A shows the surgical cut 10 to create the flap 11 in the disc annulus 6 at an annulus first end that defines the space 9. FIG. 4A shows the flap 11 being stretched across the space 9 and securely attached at a second annulus end 14 that defines the space 9 using sutures or a “stitch” 12.

In the preferred embodiments, the components, namely the instruments and devices or products used to attach the annular flap, are preferably manufactured from implant grade metallic materials such as, but not limited to, titanium and cobalt chromium alloys, nickel titanium alloys, and stainless steels. The components can also be produced from thermoplastics, composites of plastic and metal, or bioabsorbable materials. The instruments to perform the procedure may include a series of cannulae and dilators, various punches or cutters to create the flap and tools for stretching and holding the flap as explained more fully below. The manufacturing of the implants and instruments utilize standard processes but may also benefit from nano-manufacturing methods, as they develop.

It can be appreciated that the cuts made in the annulus may be made in other geometrical configurations than shown herein. The sutures or attachment devices 12 may be manufactured from polymeric implantable materials, such as polyetheretherketone (PEEK) or polyaryletherketone (PAEK) or composites thereof incorporating carbon fibers or similar materials. The instruments may be offered as a system, or kit, presented to the operating room in a case that contains various sizes and designs of components.

The sutures may be manufactured from bioabsorbable materials, including, but not limited to, polylactic acids (PLLA), polyglycolic acids (PGA), Polyglecaprone 25, Polyglactin-91 or non-absorbable materials such as, nylon and polypropylene. Synthetic or natural suture material, such as catgut, may also be employed. The sutures may be offered in sterile-packaged configuration to the operating room. The attachment of the annular flap may be accomplished by various mechanical assembly means, including but not limited to, stapling, sewing, hooks, and adhesives. The system components may be configured for application to other tissues and areas of the body.

With reference to FIG. 9, instead of using the flap 11 stretched across the space 9, a connector 15 (mechanical or natural) can be connected between the two ends 13 and 14 defining the space 9 so as to span the space 9. The attachment 17 of the connector 15 to the annulus 6 can be, for example, bio-compatible adhesive, sutures, staples, spearing, staking, male and female mating parts, fasteners, etc.

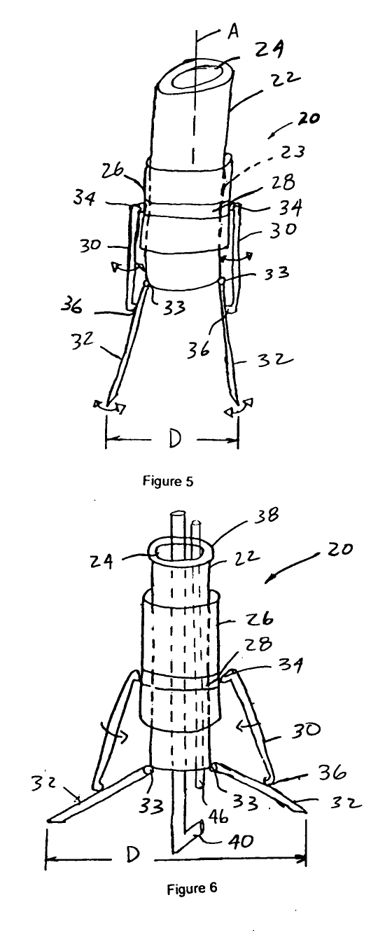

As noted above, a cutting tool is employed to create the surgical cut 10. With reference to FIGS. 5 and 6, a cutting tool is shown, generally indicated at 20, in accordance with the principles of the present invention. The cutting tool 20 includes an elongated, generally cylindrical main body 22 that includes a bore 24 there-through, the function of which will be explained below. A collar 26 is provided about at least a portion of the body 22. The collar 26 is movable along axis A of the body 22 such as, for example, via threaded engagement 23 of the collar 26 with a periphery of the body 22.

The collar 26 includes an annular groove 28 in the periphery thereof. Linkage structure, preferably in the form of a pair of links 30, is provided to couple the collar 26 to cutting structure, preferably in the form of cutting blades 32. Each blade 32 is also coupled to the body 22 via a hinge connection 33. One end of each link 30 is received in the groove 28 to define a first hinge/slop joint 34 and the other end of each link 30 is coupled with an associated blade 32 via a second hinge/slop joint 36. Thus, the hinge/slop joints 34, 36 allow for annular motion (rotation) of the collar 26. With reference to FIG. 6, as the collar 26 is moved along axis A towards the end 38 of the body 22 by rotating it in one direction with respect to the body 22 (via the threaded engagement), the cutting diameter D (distance between blades 32) of the blades 32 increases. Rotation of the collar 26 in an opposite direction decreases the cutting diameter D.

The cutting tool 20 can be used with or in place of the cannula 13 in the above-described method. Thus, to remove the herniation 8 of FIG. 2, a barb 40 (FIG. 6) is inserted into the herniation 8. The cutting tool 20 is then placed over an end of the barb 40 so that a portion of the barb 40 is within the bore 24. The collar 26 is adjusted (e.g., rotated) to set the cutting blades 32 to the appropriate cutting diameter D. The collar 26 can be coupled to a handle (not shown) that is accessible by the surgeon for rotating the collar 26. The cutting tool 20 is then rotated to make a circular cut about the herniation 8. The barb 40 and the cutting tool 20 are pulled upwardly with the barb 40 removing the herniation 8. It can be appreciated that other structure can be used instead of the barb 40 to remove the herniation 8.

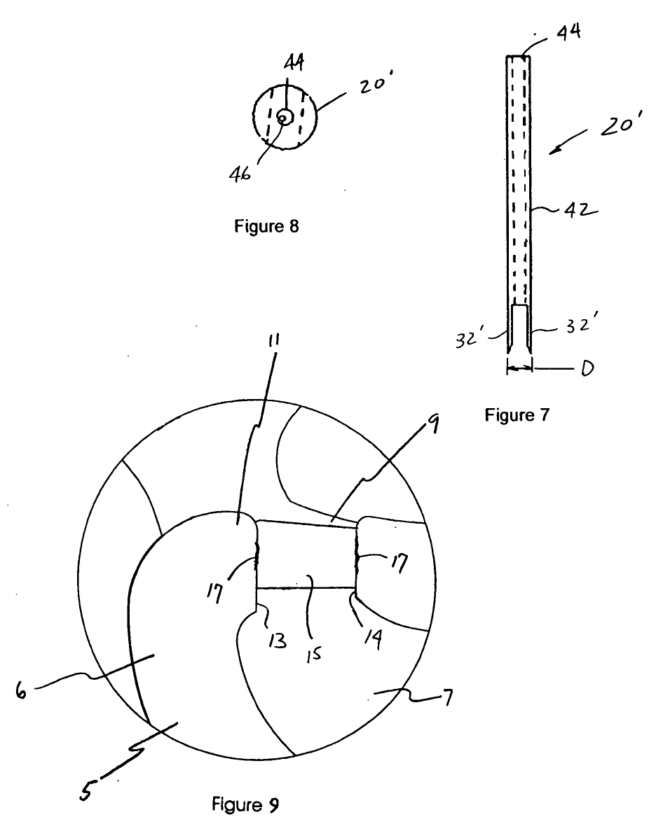

With reference to FIGS. 6 and 7, another embodiment of a cutting tool 20′ is shown. Instead of providing adjustable cutting blades as in the tool 20 of FIGS. 5 and 6, the tool 20′ has at least one cutting structure or blade 32′ of a fixed diameter D. In the embodiment, a pair of blades 32′ is provided in spaced relation such that when the tool 20′ is rotated, a cut of a certain diameter can be made. Alternatively, a circular blade of a certain diameter can be provided and can be rotated to make a cut, or can be used as a punch. The tool 20′ has an elongated body 42 with a bore 44 there-through for receiving the barb 40 (see FIG. 6) so that the damaged area (e.g., herniation) can be removed.

A plurality of tools 20′ is preferably available to the surgeon in a kit so that the surgeon can choose the appropriate diameter blade 32′. For example, six or seven tools 20′ can be provided that increase in blade diameter in 1 mm increments.

Another embodiment of the tool (not shown) includes replacing the blades 32′ shown in FIG. 7 with a laser to facilitate the cutting.

Although the embodiments have been described in a manner that the damaged area of the disc annulus is a herniation, the damaged area can be, but is not limited to, a radial tear, a tumor, and a rim lesion.

The elongated body 22 of tool 20 and the elongated body 42 of tool 20′ can each also include additional lumens (one shown at 46) that can be used for lavage (irrigation), aspiration (suction), fiber optics for illumination, and/or visualization of the surgical site.

Thus, the method and tool described herein provides an effective way to remove and repair an annulus of an intervertebral disc.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of repairing an annulus of an intervertebral disc comprising:
excising, with a cutting tool, a damaged area of the annulus, thereby creating a space in the annulus, the space being defined by first and second annulus ends,
making a partial incision at the first annulus end generally adjacent to the excised area, creating a flap of annulus,
stretching the flap across the space, and
attaching the flap to the second annulus end.

2. The method of claim 1, wherein the damaged area is a herniation.

3. The method of claim 1, wherein the damaged area is a radial tear.

4. The method of claim 1, wherein the damaged area is a tumor.

5. The method of claim 1, wherein the damaged area is a rim lesion.

6. The method of claim 1, wherein the cutting tool includes at least one cutting blade and a barb, the method further including using the cutting blade to cut the damaged area and using the barb to engage and remove the damaged area.

7. The method claim 6, wherein a pair of cutting blades is provided, the blades being in spaced relation and the step of using the cutting blade includes rotating the tool to make a generally circular cut.

8. The method of claim 7, wherein a spaced between the cutting blades is fixed.

9. The method of claim 7, wherein a space between the cutting blades is adjustable, the method including adjusting the space between the blades prior to the excising step.

10. The method of claim 1, wherein the attaching step includes using sutures to facilitate attachment of the flap.

11. The method of claim 1, wherein the attaching step includes using staples to facilitate attachment of the flap.

12. The method of claim 1, wherein the attaching step includes using a hook to facilitate attachment of the flap.

13. The method of claim 1, wherein the attaching step includes using adhesive to facilitate attachment of the flap.

14. A method of repairing an annulus of an intervertebral disc comprising:
excising, with a cutting tool, a damaged area of the annulus, thereby creating a space in the annulus, the space being defined by first and second annulus ends, and
attaching a connector between the first and second annuls ends so that the connector spans the space.

15. The method of claim 14, wherein the step of attaching includes using one of bio-compatible adhesive, sutures, staples, male and female mating parts, and fasteners to attach the connector to the first and second annulus ends.

16. The method of claim 14, wherein the cutting tool includes at least one cutting blade and a barb, the method further including using the cutting blade to cut the damaged area and using the barb to engage and remove the damaged area.

17. A tool for cutting and removing a portion of an annulus of an intervertebral disc, the tool comprising:
an elongated body having a bore there-through,
cutting structure associated with the body and constructed and arranged to cut a portion of the annulus, and
a structure movable within the bore and having an end that is constructed and arranged to engage and thereby remove the portion cut by the cutting structure.

18. The tool of claim 17, wherein the cutting structure includes a pair of blades disposed in spaced relation such that rotation of the tool creates a generally circular cut.

19. The tool of claim 18, wherein the blades are disposed a fixed distance apart to define a cutting diameter.

20. The tool of claim 19, in combination with a plurality of additional said cutting structures each associated with a said elongated body, each cutting structure having a cutting diameter different from another cutting structure.

21. The tool of claim 17, wherein the movable structure is separate and removable from the elongated body.

22. The tool of claim 18, wherein the blades are constructed and arranged so that a distance between the blades is adjustable.

23. The tool of claim 22, wherein each blade is connected to the body by a hinge, the tool further comprising:
a collar movable along an axis of the body, and
linkage structure coupling the collar with respect to the blades, the linkage structure being connected to the collar and to the blades so as to permit rotation of the collar and thereby movement of the collar along the axis,
whereby movement of the collar in one direction along the axis causes an increase in the distance between the blades and movement of the collar in a direction opposite the one direction causes the distance between the blades to decrease.

24. The tool of claim 23, wherein the collar is in threaded engagement the body permitting movement of the collar with respect to the body.

25. The tool of claim 17, further including a lumen extending through the bore of the elongated body.
